(19) United States
(12) Patent Application Publication
(10) Pub. No.: US 2024/0170095 A1
(43) Pub. Date: May 23, 2024

(54) Method and Apparatus for Reconstructing the Position of Cells in a Three-Dimensional Tissue

(71) Applicant: Robert Bosch GmbH, Stuttgart (DE)

(72) Inventors: Christopher Juerges, Renningen (DE); Damir Shakirov, Niefern-Oeschelbronn (DE)

(21) Appl. No.: 18/507,058

(22) Filed: Nov. 12, 2023

(30) Foreign Application Priority Data
Nov. 22, 2022 (DE) 10 2022 212 416.2

(51) Int. Cl. G16B 25/10

(52) U.S. Cl. CPC G16B 25/10

(57) ABSTRACT

A method for determining an assignment rule in order to merge gene expression profiles which are very similar is disclosed. The method includes (i) adjusting a model, e.g. a linear regression model, using the model to predict the gene expression profiles, (ii) calculating a cost matrix from the predictions, and (iii) applying the Hungarian algorithm to the cost matrix to obtain a new assignment rule and repeating these steps several times.

This application claims priority under 35 U.S.C. § 119 to patent application no. DE 10 2022 212 416.2, filed on Nov. 22, 2022 in Germany, the disclosure of which is incorporated herein by reference in its entirety.

The disclosure relates to a method for reconstructing the positions of cells in a three-dimensional (3D) tissue after sequencing the transcriptome of these cells, and to an apparatus which is configured to carry out the method.

### BACKGROUND

In the field of transcriptomics, it is possible to sequence the transcriptome of several individual cells. For example, methods for single-cell RNA sequencing, known as scRNA-seq methods (single cell RNA sequencing), are used for this purpose. Examples of this are 10× sequencing (10× genomics chromium sequencing) or drop sequencing.

With the scRNA-seq methods, however, the position information of the individual cells in the two-dimensional (one cell layer thick tissue section) or three-dimensional tissue examined is lost. When sequencing a tissue sample from the brain, for example, in which several different cells communicate with each other, only the transcriptomic information of the individual cells in the sample is obtained, but not the spatial information, i.e. where the cell was localized in the tissue sample and which cells were arranged adjacent to each other. Thus, scRNA-seq methods are used to obtain gene expression profiles without positional information.

However, knowing which cells are in direct contact with each other improves the quality of the information enormously, as cells influence the transcriptomic profile of adjacent cells.

This is essentially a combinatorial problem. The complexity of solving this problem is factorial, since there are n-fold many different ways of arranging the cells or their gene expression profiles so that they correspond to the correct order, wherein n is the number of gene expression profiles.

With the Slide-seq sequencing method, a first step was taken to obtain the positional information, i.e. the spatial information of individual cells in a tissue sample.

A tissue section from a single cell layer is placed on a glass slide covered with DNA-barcoded beads (small spheres in the μm range) of known positions, which can be used to deduce the original position of the RNA (ribonucleic acid) after sequencing. In this way, it is possible to deduce where the individual cells were located in the tissue section and which cells were arranged adjacent to them. Using the slide-seq method, gene expression profiles are thus obtained from cells in a single cell layer with positional information.

However, the Slide-Seq method only works with a two-dimensional tissue sample, i.e. a tissue section that is exactly one cell layer thick, so that only one layer of cells can be sequenced and mapped. Furthermore, significantly fewer transcripts can currently be detected with this method compared to the scRNA-seq methods. Applying the barcoded beads to the glass slide is also a time-consuming additional work step that increases costs and represents a potential source of error. For these reasons, it is not feasible to apply multiple slide-seq approaches for single cell layers of a three-dimensional tissue to reconstruct the 3D positions of the cells.

### Advantages of the Disclosure

The disclosure with the features set forth below has the advantage that it makes it possible to determine a potential assignment between gene expression profiles from cells of a single cell layer of a three-dimensional tissue with positional information and gene expression profiles from cells of a single cell layer of a three-dimensional tissue without positional information, without the need for subsequently added metadata, such as unique identifiers or the like.

Furthermore, the costs for sequencing are drastically reduced and there is also no need for time-consuming work cuts, for example for the application of barcoded beads, because gene expression profiles from cells of only one single cell layer with position information or a few single cell layers with position information—in particular obtained by the slide-seq method—are necessary in order to be able to assign them to gene expression profiles from cells of many single cell layers without position information—in particular obtained by scRNA-Seq methods. Thus, for example, the costly and labor-intensive slide-seq method needs to be used only once or only a few times, while at least one of the significantly less expensive scRNA-Seq methods can be used several or many times.

Another advantage of the present disclosure is that all individual single cell layers, which are sequenced for example using an scRNA-Seq method, can be sequenced together in a single sample. For this purpose, the individual single cell layers must be uniformly labeled so that it can be deduced which gene expression profile can be assigned to which single cell layer. Such barcoding of entire single cell layers is trivial in contrast to the laborious barcoding of individual cells in the slide-seq method and has already been successfully applied in several sequencing protocols.

The disclosure also has the advantage that a complete 2D and 3D cell topology of the sequenced tissue is obtained.

The spatial imaging of individual cells in a three-dimensional tissue sample, as well as the knowledge of which cells were arranged adjacent to each other, brings great advantages in the investigation of biological processes, such as the tracing of intercellular communication pathways at the transcriptional level, which in turn can provide information on the influence of drugs or diseases.

Further aspects of the disclosure are the subject matter set forth below. Advantageous further developments are the subject matter also set forth below.

### SUMMARY

The transcriptome is defined as all RNAs present in a cell at a certain point in time.

Transcriptomics can be used, for example, to investigate changes in the transcriptome that occur due to altered physiological conditions in the cell and to make statements about the activity of genes. In this way, different profiles of gene expression can be examined in order to better understand the interactions between genes and metabolic pathways in biological processes. The transcriptome can also be influenced by external factors such as drugs or diseases and can therefore be investigated under these aspects.

Gene expression is the formation of a gene product encoded by a gene, such as an RNA molecule. Gene expression consists of several individual processes, including transcription, wherein a copy of the DNA (deoxyribonucleic acid) is produced in the form of RNA.

In the context of this application, a gene expression profile is understood to be the sum of all genes expressed in a cell at a certain point in time, i.e. transcribed to mRNA (messenger ribonucleic acid).

In a first aspect, the disclosure relates to a method, in particular a computer-implemented method, for determining an assignment rule which assigns variables from a first set of first variables to respective variables from a second set of second variables.

Here, the first variables represent gene expression profiles from cells of a single cell layer of a three-dimensional tissue with position information and the second variables represent gene expression profiles from cells of a single cell layer of a three-dimensional tissue without position information.

The assignment rule can assign the first variables to the second variables in an unambiguous manner, i.e. at most one second variable is assigned to each first variable by the assignment rule and preferably vice versa. A set can be understood as a form of summary of the single variables. Preferably, the first and second sets are different sets that do not have a common variable. Preferably, an index is assigned to each of the variables of the first and second set. All indices of the first and second set could be understood as index sets. In other words, as a set whose elements index through the variables of the first or second set. The assignment rule then assigns an index from the second index set to the first index set. The assignment rule therefore describes which first variable belongs to which second variable and preferably vice versa. The assignment rule can be in the form of a list or table or similar.

The method begins with initializing the assignment rule and providing the first and second sets. The initial assignment rule can be selected randomly or as an identity assignment. Other initial assignment rules are conceivable as an alternative, e.g. a predefined, already partially correct assignment.

This is followed by repeated performance of steps a) through d) described below. The repetitions can be carried out for a predefined number of maximum repetitions or a termination criterion can be defined, wherein the repetition is terminated if the termination criterion is met. The termination criterion is, for example, a minimum change to the assignment rule.
a) Creating a data set which contains the first variables and their respective second variables assigned according to the assignment rule. The data set can also be referred to as a training data set, wherein the assigned second variables are so-called “labels” of the first variables. It should be noted that this step can be optional, since the subsequent steps using this data set essentially require only the information of the current assignment rule between the first and second variables, which can be provided either by the data set or by a current assignment rule. The current assignment rule is the assignment rule that exists for the current repetition of steps a) through d), i.e. the assignment rule that was used when the most recent creation of the data set was performed.
b) Training a machine learning system in such a way that the machine learning system determines the respectively assigned second variables of the data set depending on the first variables. Training can be understood to mean that parameters of the machine learning system are adjusted so that the predictions determined by the machine learning system are as close as possible to the second variables (“labels”) of the data set. The optimization can be carried out with regard to a cost function. The cost function preferably characterizes a mathematical difference between the outputs of the machine learning system and the labels. The optimization is preferably carried out using a gradient descent method. The machine learning system can be one or a plurality of decision trees, a neural network, a support vector machine, or similar. Training can be carried out until a further improvement in the machine learning system during training is negligible, i.e. a second termination criterion is met.
c) Determining a cost matrix, wherein entries of the cost matrix characterize a distance between the prediction of the machine learning system and the second variables according to the assignment rule, in particular between the predictions of the machine learning system and all variables of the second set. The distance can be determined using a L₂ norm. Other spacing dimensions are also conceivable. The cost matrix can be structured in such a way that rows and columns are each assigned to a first variable or the prediction of the machine learning system depending on the first variable and a second variable, wherein the entries characterize the distance between the respective assigned variables of the rows and columns. The entries that do not lie on the diagonal of the cost matrix can be regarded as transportation costs that must be incurred in order to assign the first variables to the respective second variables of the corresponding rows/columns contrary to the assignment rule.
d) Optimization of the assignment rule depending on the cost matrix so that the assignment rule generates minimum total costs based on the entries in the cost matrix. Total costs correspond to a sum of those entries of the cost matrix that are required when performing an assignment of the variables of the first set to the second set according to the current assignment rule from the cost matrix. In other words, the sum of the entries that are selected from the cost matrix depending on the assignment rule is optimized, in particular minimized. It should be noted that the entries are selected depending on the assignment rule in such a way that the entries of the respective column and row of the cost matrix are selected according to the assignment rule, which are assigned to the first and second variables that are assigned to each other according to the assignment rule.

The assignment rule determined in the last repetition of step d) is the final assignment rule, which is output in an optional step.

For example, a three-dimensional tissue is cut into many tissue sections, each one cell layer thick—referred to here as single cell layers.

The gene expression profiles of these single cell layers are obtained, for example, using different methods so that, on the one hand, gene expression profiles with position information of the cells in the respective single cell layer are obtained and, on the other hand, gene expression profiles without position information of the cells of other single cell layers are obtained.

The assignment rule can now assign the gene expression profiles from cells of a single cell layer of the three-dimensional tissue without positional information to the gene expression profiles from cells of a single cell layer of the three-dimensional tissue with positional information in an unambiguous manner. The underlying principle here is that the gene expression profile of specific genes is very similar in adjacent cells. In addition, the overall change in cell topology is limited from one single cell layer to the next adjacent single cell layer in the three-dimensional tissue, so that their topology or spatial arrangement can be predicted with the aid of the preceding single cell layer.

The variables can be scalars or vectors of one or more genes. Preferably, the first and second variables are each one or a plurality of measurement results from one measurement of genes or from a plurality of different measurements of genes, each performed on one of a plurality of objects. This means that each variable is assigned to one of the objects. In the step of creating the data set, only a predeterminable number of measurement results of the plurality of measurement results can be used for the second variables. The assignment rule can specify which first and second variables are measurement results of the same object.

It is proposed that the optimization of the assignment rule is carried out using a cost minimization algorithm under the given cost matrix. For example, optimization using a Hungarian algorithm applied to the cost matrix is conceivable. The Hungarian method, also known as the Kuhn-Munkres algorithm, is an algorithm for solving weighted assignment problems. Alternatively, a greedy implementation of the algorithm can also be used to minimize costs.

Furthermore, it is proposed that the machine learning system is a regression model which determines the second variables depending on the first variables and parameters of the regression model, wherein the parameters of the regression model are adjusted during training.

Regression is used to model relationships between a dependent (often also explained variable) and one or more independent variables (often also explanatory variables). Regression is able to parameterize a more complex function so that it best represents data according to a certain mathematical criterion. For example, the ordinary least squares method calculates a unique straight line (or hyperplane) that minimizes the sum of squares of deviations between the true data and this line (or hyperplane), i.e. the residual sum of squares.

Linear regression has proven to be particularly effective for the machine learning system in finding the best assignment rule. This is because it is based on a linear relationship, which is a reasonable assumption for the assignment of gene expression profiles. Linear regression is a special case of regression. In linear regression, a linear function is assumed. This means that only those correlations are used where the dependent variable is a linear combination of the regression coefficients (but not necessarily of the independent variables).

Preferably, there are as many gene expression profiles without position information as gene expression profiles with position information. This results, for example, from the fact that if second gene expression profiles of a second single cell layer are initially present without positional information and these first gene expression profiles of a first single cell layer are assigned with positional information, the second gene expression profiles of the second single cell layer are also those with positional information after the assignment. In a further step, third gene expression profiles of a third single cell layer without positional information can then be assigned to the second gene expression profiles of the second single cell layer, and so on.

Furthermore, it is proposed that the gene expression profiles with positional information are obtained using the slide-seq method or a comparable method and the gene expression profiles without positional information are obtained using an scRNA-seq method.

The Slide-Seq method is based on the attachment of RNA-binding, DNA-barcoded microbeads to a rubber-coated glass slide. The microbeads used are DNA-barcoded, 10 μm microparticles, each of which contains DNA only once, so that unique identification is possible.

The microbeads are assigned to their spatial position using SOLID sequencing (Sequencing by Oligonucleotide Ligation and Detection). A tissue section in the form of a single cell layer is then transferred to the glass slide. The RNA released from the tissue section is bound by the microbeads, resulting in a 3′-end barcoded RNA sequencing library. The bound RNA is first reverse transcribed into cDNA (complementary DNA), amplified and finally sequenced. The localization of the transcript is determined by the barcode oligonucleotide sequence of the microbead that captured it. In this way, a gene expression profile is obtained from cells of a single cell layer with positional information.

The protocols of the various scRNA-seq methods have the following procedure in common: Cells are separated from a tissue, for example a tissue section in the form of a single cell layer, and incubated with one microbead each. These microbeads are loaded with numerous oligonucleotides consisting of primers for cDNA (complementary DNA) synthesis and individual barcodes. A primer is an oligonucleotide with a known nucleotide sequence that serves as a starting point for DNA-replicating enzymes such as DNA polymerase. The cDNAs are amplified and finally sequenced.

In this way, a gene expression profile is obtained from cells in a single cell layer without positional information.

It is also proposed that the method be used to deduce the original position of each cell in a three-dimensional tissue.

In a further advantageous embodiment of the method, after repeating steps a) through c) any number of times, a further first variable, i.e. a gene expression profile with positional information, is added to the set of first variables. The addition of a further first variable to the set of first variables can take place according to a predefined pattern, for example after a certain number of repetitions of steps a) through c). Alternatively, an additional first variable can be added once or at random, according to a predefined or non-defined pattern.

By adding at least one further first variable to the set of first variables, the robustness of the convergence behavior of the method is improved, whereby the assignment more reliably approximates the theoretically correct solution and thus ultimately a more reliable and more accurate assignment rule can be determined.

In further aspects, the disclosure relates to an apparatus and to a computer program, which are each configured to perform the aforementioned methods, and to a machine-readable storage medium on which said computer program is stored.

### BRIEF DESCRIPTION OF THE DRAWINGS

Embodiments of the disclosure are explained in greater detail below with reference to the accompanying drawings. In the drawings:

FIG. 1 schematically shows an overview of how the first and second variables are obtained and how the method according to the disclosure is used to determine the assignment rule for the first and second variables,

FIG. 2 schematically shows an embodiment example of a flow chart of the disclosure, and

FIG. 3 schematically shows a training apparatus.

### DETAILED DESCRIPTION

When examining the transcriptome of cells in a three-dimensional tissue using scRNA-Seq methods, a gene expression profile is obtained in which the expressed genes can be precisely assigned to the respective cells. However, the position information of the individual cells is lost, so that it is not possible to deduce which cell was arranged at which spatial position in the three-dimensional tissue and which cells were adjacent to it, i.e. positioned adjacent to it.

The slide-seq method, on the other hand, is used to obtain a gene expression profile of a tissue section one cell layer thick (hereinafter referred to as a single cell layer), in which the expressed genes can be precisely assigned to the respective cells and the exact position of the respective cells in the single cell layer of the three-dimensional tissue can also be deduced. However, it is possible to reconstruct the position of the cells only in two-dimensional tissue, but not in three-dimensional tissue.

One task of the disclosure is to be able to deduce the spatial arrangement of gene expression profiles of individual cells of a three-dimensional tissue after sequencing of the expressed genes and, in particular, to obtain knowledge of which cells were arranged adjacent to one another in the three-dimensional tissue.

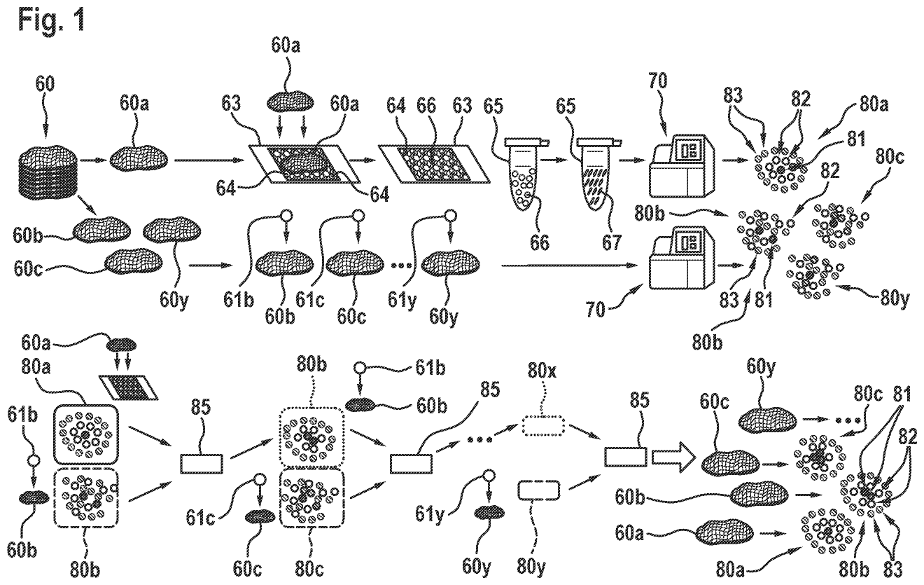

To this end, the disclosure proposes the embodiment example shown in FIG. 1.

A three-dimensional tissue 60 is cut into single tissue cell layers 60a, 60b, 60c, . . . 60y. The upper path represents a slide-seq method. The single cell layer 60a is applied to a glass slide 63, which is covered with DNA-barcoded beads 64 of known positions.

The mRNAs of the cells of the single cell layer 60a attach to the DNA-encoded beads, resulting in a barcoded RNA sequencing library 66. This is reverse transcribed and amplified. Reverse transcription and amplification are carried out, for example, in a microreaction vessel 65. The amplicons 67 are finally subjected to sequencing by means of a sequencing device 70, whereby a gene expression profile 81, 82, 83 is obtained for each cell of the single cell layer 60a with information on the exact position of the cell in the single cell layer 60a. The position of the cells in the single cell layer 60a is deduced via the corresponding barcode of the DNA-encoded beads. Thus, a first gene expression profile layer 80a is obtained in which the spatial arrangement of the individual gene expression profiles 81, 82, 83 in the single cell layer 60a is known. In FIG. 1, the knowledge of the position of the individual gene expression profiles 81, 82, 83 and thus of the cells in the single cell layer 60a is to be indicated by the fact that first gene expression profiles 81 are arranged on the inside, second gene expression profiles 82 in the middle and third gene expression profiles 83 on the outside of the first gene expression profile layer 80a. It should be noted here that the first 81, second 82 and third gene expression profiles 83 are not each intended to represent a conglomerate of gene expression profiles, but are the gene expression profiles of the individual cells of the tissue single cell layers 60a. The number of gene expression profiles 81, 82, 83 thus corresponds to the number of cells in the single cell layer 60a. The subdivision into first 81, second 82 and third 83 gene expression profiles in FIG. 1 is merely intended to provide a visual illustration that there can be different cell types in the single cell layer 60a, to which similar cell types of a further gene expression profile 60b can then be attached or assigned in further steps (described below in the lower path of FIG. 1).

The second path maps an scRNA sequencing method. The scRNA-Seq can be performed according to known protocols and is not described in more detail in FIG. 1. For example, the individual single cell layers 60b, 60c . . . . 60y can all be sequenced together in a single sample. For this purpose, the single cell layers 60b, 60c, . . . 60y are each provided with a barcode or marker 61b, 61c, . . . 61y, so that it can be deduced which gene expression profile is to be assigned to which single cell layer. Such barcoding of single cell layers can also be carried out according to known protocols. After reverse transcription and amplification, sequencing is carried out using a sequencing device 70. A gene expression profile 81, 82, 83 is obtained for each cell, which can be assigned to the respective single cell layer 60b, 60c, . . . 60y via the barcode. However, the position information of the individual gene expression profiles 81, 82, 83 within the single cell layers 60b, 60c, . . . 60y is lost. Thus, second 80b, third 80c and y-th gene expression profile layers 80y are obtained in which the spatial arrangement of the individual gene expression profiles 81, 82, 83 is not known. This is indicated in FIG. 1 by the fact that the first 81, second 82 and third gene expression profiles 83 are intermixed and randomly arranged in their respective gene expression profile layers 80b, 80c, . . . 80y.

In order to reconstruct the position of the individual gene expression profiles 81, 82, 83 of the individual cells in the respective gene expression profile layers 80b, 80c, . . . 80y, an assignment algorithm is proposed which consists of an alternating sequence of optimization of regression parameters (when regressing from gene expression profiles 81, 82, 83 with position information to gene expression profiles 81, 82, 83 without position information) and subsequent optimization of the assignment of gene expression profiles 81, 82, 83. The current assignment of the gene expression profiles 81, 82, 83 without position information is used as a “regression label” in each iteration.

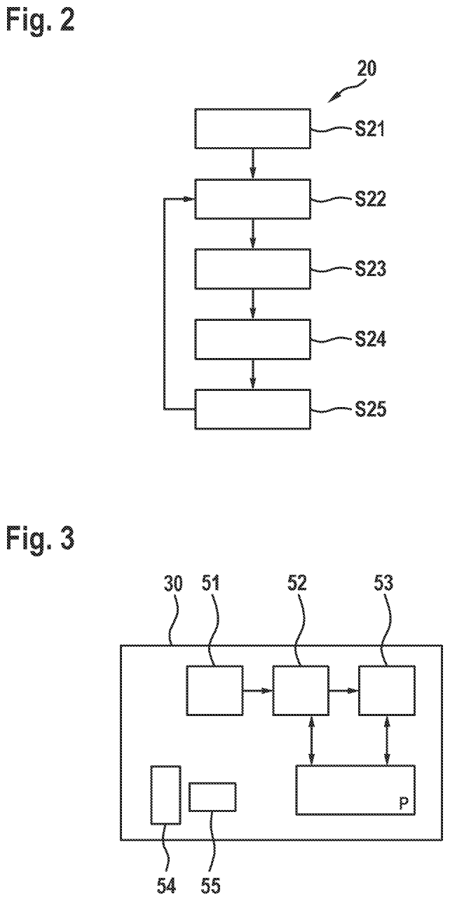

Thus, in the path described in FIG. 1 below, a first gene expression profile layer 80a from gene expression profiles 81, 82, 83 with positional information and a second gene expression profile layer 80b comprising gene expression profiles 81, 82, 83 without positional information are subjected to the method according to the disclosure for determining an assignment rule, as also shown and described in flow chart 20 of FIG. 2.

Reference number 85 is intended to represent the technical system which is configured to carry out the method according to flow chart 20 of FIG. 2. The first variables, i.e. gene expression profiles from cells of a single cell layer of a three-dimensional tissue with positional information, and the second variables, i.e. gene expression profiles from cells of a single cell layer of a three-dimensional tissue without positional information, are entered into the technical system 85 and a proposal for an assignment or a spatial arrangement of the gene expression profiles of the individual cells without positional information is obtained therefrom.

Hereby, the gene expression profiles 81, 82, 83 of the second gene expression profile layer 80b without position information are spatially assigned to the gene expression profiles 81, 82, 83 of the first gene expression profile layer 80a with position information due to the high similarity of adjacent gene expression profiles 81, 82, 83. In this way, the gene expression profiles 81, 82, 83 of the second gene expression profile layer 80b finally become those with positional information.

The gene expression profiles 81, 82, 83 of the second gene expression profile layer 80b with position information and gene expression profiles 81, 82, 83 of a third gene expression profile layer 80c without position information are now again assigned to one another in the technical system 85 of the method according to the disclosure, so that finally the gene expression profiles 81, 82, 83 of the third gene expression profile layer 80c are also those with position information. In this way, any number of gene expression profiles 81, 82, 83 from gene expression profile layers 80x with position information and those from gene expression profile layers 80y without position information can be assigned to each other. Finally, all gene expression profiles 81, 82, 83 without positional information are assigned such information by the method according to the disclosure, so that the position at which each individual cell was originally located in the three-dimensional tissue 60 is reconstructed.

Here, gene expression profiles 81, 82, 83 of a single cell layer 60a lying on the outside of the three-dimensional tissue 60 can be the one with positional information. Alternatively, gene expression profiles 81, 82, 83 of a single cell layer 60b, 60c, 60x arranged centrally in the three-dimensional tissue 60 can also be those with positional information. Furthermore, gene expression profiles 81, 82, 83 of several single cell layers with position information can be available. Particularly advantageously, a gene expression profile layer comprising gene expression profiles 81, 82, 83 with position information is provided for each arbitrary number of gene expression profile layers comprising gene expression profiles 81, 82, 83 without position information. The addition of gene expression profile layers 80a-y from gene expression profiles 81, 82. 83 with position information can be carried out once or several times according to a predefined pattern or several times according to any pattern, in particular one that is not predetermined.

In the proposed method for determining an assignment rule, second variables, namely gene expression profiles without position information, are turned into first variables, namely gene expression profiles with position information. In the course of the method, therefore, some variables are removed from the set of first variables and transferred to the set of second variables. This means that the set of the first and second variables changes during the method.

The disclosure also uses a cost-minimizing algorithm that can determine an optimal assignment under a predefined cost matrix. In order to construct a suitable cost matrix, a regression error is used when calculating a suitable distance measure (e.g. L₂ norm) between the prediction of the gene expression profile of a trained regressor and the regression label. Based on this cost matrix, the algorithm rearranges the expression profiles without position information in such a way that the regression loss is minimized. Depending on the characteristics of the data, the regressor or regression model can be freely selected (e.g. linear regression for linear dependencies).

FIG. 2 schematically shows a flow chart 20 of a method for determining an assignment rule which assigns a gene expression profile 81, 82, 83 from cells of a single cell layer 60a, 60b, 60c, . . . 60y of a three-dimensional tissue 60 without positional information to a gene expression profile 81, 82, 83 from cells of a single cell layer 60a, 60b, 60c, . . . 60y of a three-dimensional tissue 60 with positional information which is most similar to the latter itself. After completion of the method, an assignment rule should be available which assigns the gene expression profiles from cells of a single cell layer without position information to those gene expression profiles from cells of a single cell layer with position information which are most similar to these. The original arrangement of the individual cell layers in the three-dimensional tissue can then be reconstructed and the position of individual cells in the three-dimensional tissue can be deduced.

The method begins with step S21. The assignment rule is initialized in this step. Furthermore, in this step, the gene expression profiles from cells of a single cell layer of a three-dimensional tissue with positional information (gene expression profiles with positional information) and the gene expression profiles from cells of a single cell layer of a three-dimensional tissue without positional information (gene expression profiles without positional information) are provided.

Step S22 is then performed. A training data set is created which contains the gene expression profiles with position information and their respective gene expression profiles without position information assigned according to the assignment rule.

After step S22 has been completed, step S23 follows. In this step, a regressor f is trained so that, depending on the gene expression profiles with position information (GEP with location), the regressor determines the respective assigned gene expression profiles without position information (GEP without location) according to the training data set: (GEP mit Ort)=GEP ohne Ort. The regressor f can be a linear regression model. The regressor is trained in a known manner, e.g. by minimizing a regression error on the training data set by adjusting the parameters of the regressor f.

After the regressor has been trained, step S24 follows. A cost matrix is created here. The rows and columns are each assigned to a gene expression profile with position information and a gene expression profile without position information. The entries of the cost matrix are determined, for example, by means of a L₂ norm between the prediction of the regressor depending on the corresponding gene expression profile with position information of the respective row and the corresponding gene expression profile without position information of the respective column from the training data and stored in the cost matrix.

After step S24 has been completed, the assignment rule is optimized in step S25. The optimization is performed by applying the Hungarian algorithm to the cost matrix in order to obtain an improved assignment rule based on the cost matrix.

If a termination criterion is not met, steps S22 to S25 are carried out again. The termination criterion can be a predefined maximum number of repetitions.

If the termination criterion is met, the method is completed and the assignment rule can be output.

In an optional step following step S25, the position of the gene expression profiles—and thus of the respective cells which have the corresponding gene expression profile—in the three-dimensional tissue is reconstructed by means of the assignment rule. The gene expression profiles with position information can be determined using the assignment rule, starting with the gene expression profiles without position information and working backwards.

FIG. 3 schematically shows an apparatus 30 for carrying out the method according to FIG. 2.

The apparatus comprises a provider 51 that provides the training data set according to step S22. The training data is then fed to the regressor 52, which uses it to determine output variables. Output variables and training data are fed to an evaluator 53, which uses them to determine updated parameters of the regressor 52, which are transmitted to the parameter memory P and replace the current parameters there. The evaluator 53 is configured to carry out step S23.

The steps performed by the apparatus 30 can be implemented as a computer program stored on a machine-readable storage medium 54 and performed by a processor 55.

The term “computer” comprises any device for processing predeterminable calculation rules. These calculation rules can be in the form of software, or in the form of hardware, or also in a mixed form of software and hardware.

What is claimed is:

1. A method for determining an assignment rule which assigns first variables from a first set of first variables to second variables from a second set of second variables, wherein the first variables are gene expression profiles from cells of a single cell layer of a three-dimensional tissue with positional information and wherein the second variables are gene expression profiles from cells of a single cell layer of a three-dimensional tissue without positional information, the method comprising:
initializing the assignment rule and providing the first and second set; and
repeating performance of the following steps a) through c):
a) training a machine learning system in such a way that the machine learning system determines the second variables assigned in each case in accordance with the assignment rule as a function of the first variables;
b) determining a cost matrix, wherein entries of the cost matrix characterize distances between predictions of the machine learning system depending on the first variables and the second variables; and
c) optimizing the assignment rule depending on the cost matrix so that an assignment of the first variables to the second variables according to the assignment rule generates minimum total costs based on the entries of the cost matrix.

2. The method according to claim 1, wherein step c) is carried out by way of a Hungarian algorithm or a greedy implementation.

3. The method according to claim 2, wherein:
the cost matrix is quadratic, and
if a number of the first and second variables do not match, then empty entries of the cost matrix are filled with a largest value of the entries of the cost matrix.

4. The method according to claim 1, wherein the machine learning system is a regression model which determines the second variables depending on the first variables and parameters of the regression model.

5. The method according to claim 1, wherein the gene expression profiles with positional information are obtained by the slide-seq method and the gene expression profiles without positional information are obtained by an scRNA-seq method.

6. The method according to claim 1, wherein the original position of each cell in the three-dimensional tissue is deduced by way of the assignment rule.

7. The method according to claim 1, wherein after performing a repetition of steps a) through c), a further first variable is added to the set of first variables.

8. An apparatus which is configured to carry out the method according to claim 1.

9. A computer program comprising instructions which, when the program is performed by a computer, cause the computer to carry out the method according to claim 1.

10. A machine-readable storage medium on which the computer program according to claim 9 is stored.
